(19) United States
(12) Patent Application Publication
(10) Pub. No.: US 2005/0132507 A1
(43) Pub. Date: Jun. 23, 2005

(54) Tertiary p-phenylenediamines, dyes compositions, and dyeing processes

(76) Inventors: Eric Metais, St Leu la Forêt (FR); Xavier Radisson, Paris (FR); Stephane Sabelle, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/997,893

(22) Filed: Nov. 29, 2004

Related U.S. Application Data
(60) Provisional application No. 60/527,779, filed on Dec. 9, 2003.

(30) Foreign Application Priority Data
Nov. 28, 2003 (FR) 03 14021

(51) Int. Cl. A61K 7/13

(52) U.S. Cl. 8/406

(57) ABSTRACT

The disclosure provides para-phenylenediamine compounds, dye compositions comprising the para-phenylenediamines, and processes for dyeing keratin fibers using the compositions.

This application claims the benefit of U.S. Provisional Application No. 60/527,779, filed Dec. 9, 2003, which is incorporated herein by reference.

The present disclosure provides novel para-phenylenediamines and dye compositions comprising them. The disclosure also provides dye compositions comprising certain para-phenylenediamines and processes and kits for dyeing keratin fibers using the compositions.

It is known practice to dye keratin fibers, and in particular human hair, with dye compositions containing oxidation dye precursors, which are generally known as oxidation bases. These oxidation bases are colorless or weakly colored compounds, which, when combined with oxidizing products, may give rise to colored compounds by a process of oxidative condensation. It is also known that the shades obtained with these oxidation bases may be varied by combining them with couplers or coloration modifiers. The variety of molecules used as oxidation bases and couplers allows a wide range of colors to be obtained.

The oxidation dyeing process comprises applying to the keratin fibers bases or a mixture of bases and couplers with aqueous hydrogen peroxide solution as the oxidizing agent, leaving the mixture to act, and then rinsing the fibers. This process, which is generally performed at basic pH, makes it possible to simultaneously dye and lighten the fibers. In practice, this means that the final color obtained may be lighter than the original color.

Further, this type of coloration typically must satisfy a certain number of requirements. Ideally, it has no toxicological drawbacks, is able to produce shades of the desired intensity, and shows good resistance to external agents such as light, bad weather, washing, permanent-waving, perspiration, and rubbing.

In addition, it is desirable for oxidation dyes to be able to cover white hair and to be as unselective as possible, i.e., produce the smallest possible differences in coloration along the same keratin fiber, which may in fact be differently sensitized (i.e., damaged) between its end and its root. Finally, oxidation dyes should have good chemical stability in their formulations and have a good toxicological profile.

Para-phenylenediamine and para-tolylenediamine are widely used oxidation bases in the field of hair dyeing. Varied shades may be obtained with these oxidation bases, including when they are used with oxidation couplers.

However, there is a need for new oxidation bases that have better toxicological profiles than para-phenylenediamine and para-tolylenediamine, while at the same time imparting to the hair excellent properties in terms of at least one of: color intensity, variety of shades, color uniformity, and resistance to external agents.

This need is met by the various embodiments of the present invention. In a first aspect, compounds of formula (I): 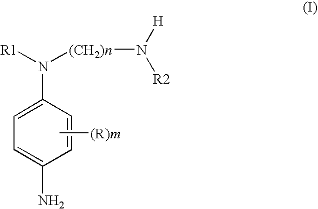
and addition salts thereof are provided, wherein:
R is independently chosen from linear and branched C₁-C₄ alkyl radicals; and linear and branched C₁-C₄ alkoxy radicals;
m is an integer ranging from 0 to 4;
R₁ is chosen from linear and branched C₁-C₄ alkyl radicals; linear and branched C₂-C₄ hydroxyalkyl radicals; and linear and branched (C₁-C₄)alkoxy(C₂-C₄)alkyl radicals;
R₂ is chosen from a hydrogen atom; linear and branched C₁-C₄ alkyl radicals; linear and branched C₂-C₄ hydroxyalkyl radicals; and linear and branched (C₁-C₄)alkoxy(C₂-C₄)alkyl radicals; and
n is an integer ranging from 2 to 6.

In another aspect, the disclosure provides dye compositions comprising, in a medium suitable for dyeing keratin fibers, such as human keratin fibers, at least one compound of formula (I) and addition salts thereof.

In another aspect, the disclosure provides processes for dyeing keratin fibers, such as human keratin fibers, using the dye compositions.

In still another aspect, the disclosure provides multi-compartment devices for performing the processes for dyeing keratin fibers.

Other characteristics and advantages of the present disclosure will be clear from reading the description and the examples that follow.

The compound of formula (I) will first be described.

In some embodiments, each radical R, which may be identical or different, is a C₁-C₂ alkyl radical, for example, a methyl radical.

In some embodiments, m is an integer ranging from 0 to 2, such as 0 or 1.

In some embodiments, R₁ is chosen from a C₁-C₂ alkyl radical, a C₂ hydroxyalkyl radical, and a (C₁-C₄)alkoxy(C₂)alkyl radical. In certain embodiments, R₁ is chosen from methyl, ethyl and hydroxyethyl radicals.

In some embodiments, R₂ is chosen from a hydrogen atom, a C₁-C₂ alkyl radical, a C₂ hydroxyalkyl radical, and a (C₁-C₄)alkoxy(C₂)alkyl radical. For example, R₂ is chosen from a hydrogen atom, a methyl radical, and a hydroxyethyl radical. In other embodiments, R₂ is other than hydrogen, for example, chosen from a hydrogen atom; linear and branched C₁-C₄ alkyl radicals; linear and branched C₂-C₄ hydroxyalkyl radicals; and linear and branched (C₁-C₄)alkoxy(C₂-C₄)alkyl radicals.

In certain embodiments, the compound of formula (I) is chosen from the following compounds and addition salts thereof:

| | |
|---|---|
| 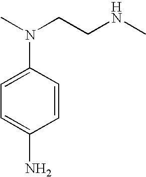 | N-Methyl-N-(2-methylaminoethyl)benzene-1,4-diamine |
| 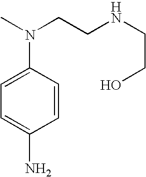 | 2-{2-[(4-Aminophenyl)methylamino]ethylamino}ethanol |
| 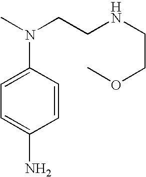 | N-[2-(2-Methoxyethylamino)ethyl]-N-methylbenzene-1,4- diamine |
| 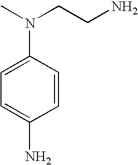 | N-(2-Aminoethyl)-N-ethylbenzene-1,4-diamine |
| 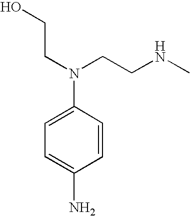 | 2-[(4-Aminophenyl)(2-methylaminoethyl)amino]ethanol |
| 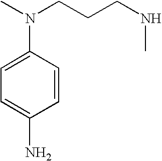 | N-Methyl-N-(3-methylaminopropyl)benzene-1,4-diamine |
| 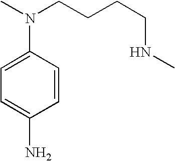 | N-Methyl-N-(4-methylaminobutyl)benzene-1,4-diamine |
| 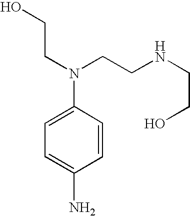 | 2-{(4-Aminophenyl)[2-(2-hydroxy- ethylamino)ethyl]amino}ethanol |
| 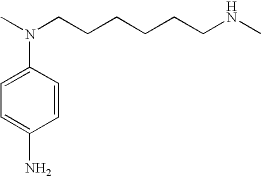 | N-Methyl-N-(6-methylaminohexyl)benzene-1,4-diamine |
| 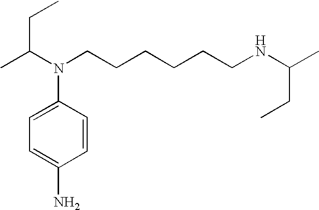 | N-sec-Butyl-N-(6-sec-butylaminohexyl)benzene-1,4- diamine |
| 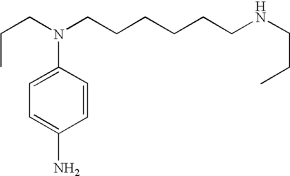 | N-Propyl-N-(6-propylaminohexyl)benzene-1,4-diamine |
| 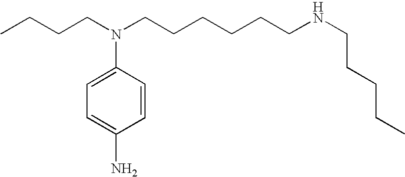 | N-Butyl-N-(6-butylaminohexyl)benzene-1,4-diamine |
| 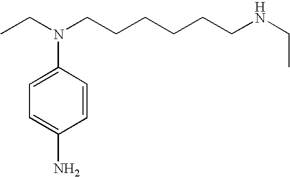 | N-Ethyl-N-(6-ethylaminohexyl)benzene-1,4-diamine |
| 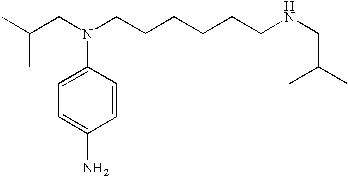 | N-Isobutyl-N-(6-isobutylaminohexyl)benzene-1,4-diamine |
| 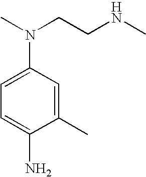 | 2,N′4′-Dimethyl-N4-(2-methylaminoethyl)benzene-1,4- diamine |
| 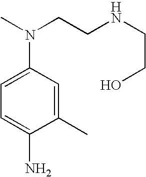 | 2-{2-[(4-Amino-3-methylphenyl)- methylamino]ethylamino}ethanol |
| 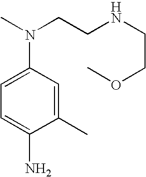 | N-4-[2-(2-Methoxyethylamino)ethyl]-2,N-4- dimethylbenzene-1,4-diamine |
| 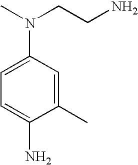 | N-4-(2-Aminoethyl)-N-4-ethyl-2-methylbenzene-1,4- diamine |
| 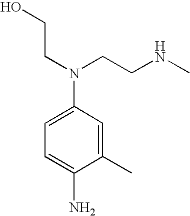 | 2-[(4-Amino-3-methylphenyl)(2- methylaminoethyl)amino]ethanol |
| 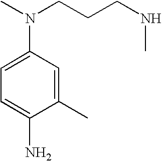 | 2,N-4-Dimethyl-N-4-(3-methylaminopropyl)benzene-1,4- diamine |
| 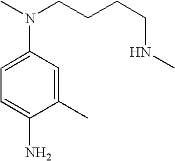 | 2,N-4-Dimethyl-N-4-(4-methylaminobutyl)benzene-1,4- diamine |
| 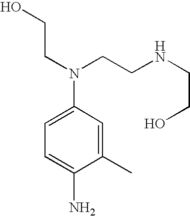 | 2-{(4-Amino-3-methylphenyl)[2-(2- hydroxyethylamino)ethyl]amino}ethanol |
| 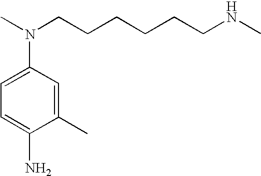 | 2,N-4-Dimethyl-N-4-(6-methylaminohexyl)benzene-1,4- diamine |
| 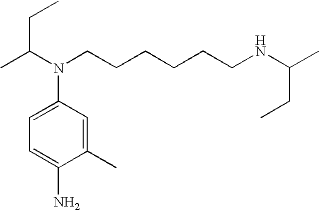 | N-4-sec-Butyl-N-4-(6-sec-butylaminohexyl)-2- methylbenzene-1,4-diamine |
| 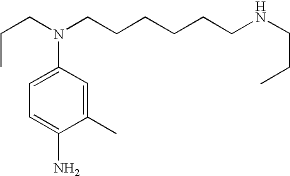 | 2-Methyl-N-4-propyl-N-4-(6-propylaminohexyl)benzene- 1,4-diamine |
| 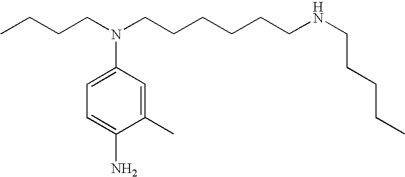 | N-4-Butyl-N-4-(6-butylaminohexyl)-2-methylbenzene-1,4- diamine |
| 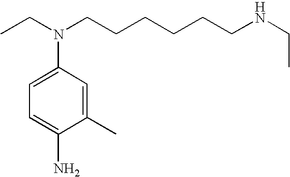 | N-4-Ethyl-N-4-(6-ethylaminohexyl)-2-methylbenzene-1,4- diamine |
| 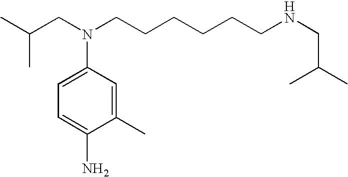 | N-4-Isobutyl-N-4-(6-isobutylaminohexyl)-2- methylbenzene-1,4-diamine |

In general, the addition salts of the compounds of formula (I) may be chosen from acid addition salts, such as the hydrochlorides, hydrobromides, sulfates, citrates, succinates, tartrates, lactates, tosylates, benzenesulfonates, phosphates, and acetates.

The compounds of formula (I) may be synthesized according to the following reaction scheme:

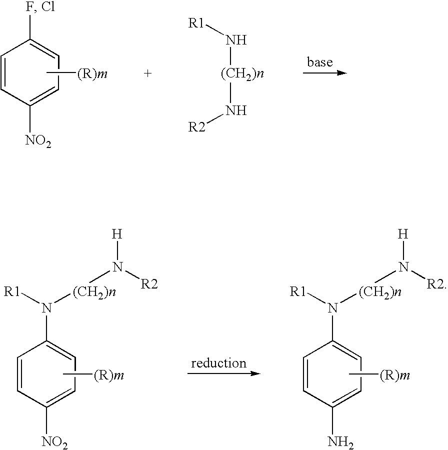

The first step is similar to the syntheses described in “Synthesis”, 1990 (12), 1147-1148 and “Synth. Commun.” 1990, 20 (22), 3537-3545.

The second step is a standard reduction step described, for example, in patent application DE 4,241,532 and “J. Heterocycl. Chem.” 1992, 29 (6) 1513-1517.

The disclosure also provides dye compositions comprising, in a medium suitable for dyeing keratin fibers, such as human keratin fibers, at least one compound of formula (I) or addition salts thereof as described above. In such oxidation dyeing compositions, the compound of formula (I) or its addition salts may be present as the oxidation base.

In one embodiment, the amount of the compound of formula (I) or addition salts thereof ranges from 0.001% to 10% by weight relative to the total weight of the dye composition, such as from 0.005% to 6% by weight relative to the total weight of the dye composition.

The dye compositions may also comprise at least one additional oxidation base optionally combined with at least one coupler, chosen from those conventionally used for dyeing keratin fibers.

The at least one additional oxidation base may be chosen from para-phenylenediamines other than those of the compound of formula (I), bis(phenyl)alkylenediamines, para-aminophenols, bis-para-aminophenols, ortho-aminophenols, heterocyclic bases, and addition salts thereof.

Para-phenylenediamines useful herein include, for example, para-phenyl-enediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenyl-enediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene, 3-hydroxy-1-(4′-aminophenyl)pyrrolidine, and acid addition salts thereof.

For example, the para-phenylenediamines may be chosen from para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-β-acetylaminoethyloxy-para-phenylenediamine, and the acid addition salts thereof.

The bis(phenyl)alkylenediamines useful herein may be chosen from, for example, N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)-tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diamino-phenoxy)-3,6-dioxaoctane, and acid addition salts thereof.

The para-aminophenols may be chosen from, for example, para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethyl)phenol, 4-amino-2-(β-hydroxyethylaminomethyl)phenol, 4-amino-2-fluorophenol, and acid addition salts thereof.

The ortho-aminophenols may be chosen from, for example, that 2-aminophenol, 2-amino-5-methylphenol, 2-amino-6-methylphenol, 5-acetamido-2-aminophenol, and acid addition salts thereof.

The heterocyclic bases may be chosen from, but are not limited to, pyridine derivatives, pyrimidine derivatives, pyrazole derivatives and acid addition salts thereof.

The pyridine derivatives may be chosen from, for example, the compounds described in patents GB 1,026,978 and GB 1,153,196, as well as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine, and acid addition salts thereof.

Other pyridine oxidation bases that may be used include 3-amino-pyrazolo[1,5-a]pyridine oxidation bases and the addition salts thereof described, for example. in patent application FR 2,801,308. Mention may be made, for example, of pyrazolo[1,5-a]pyrid-3-ylamine; 2-acetylaminopyrazolo[1,5-a]pyrid-3-ylamine; 2-morpholin-4-ylpyrazolo[1,5-a]pyrid-3-ylamine; 3-aminopyrazolo[1,5-a]pyridine-2-carboxylic acid; 2-methoxypyrazolo[1,5-a]pyrid-3-ylamine; (3-aminopyrazolo[1,5-a]pyrid-7-yl)methanol; 2-(3-aminopyrazolo[1,5-a]pyrid-5-yl)ethanol; 2-(3-aminopyrazolo[1,5-a]pyrid-7-yl)ethanol; (3-aminopyrazolo[1,5-a]pyrid-2-yl)methanol; 3,6-diaminopyrazolo[1,5-a]pyridine; 3,4-diaminopyrazolo[1,5-a]pyridine; pyrazolo[1,5-a]pyridine-3,7-diamine; 7-morpholin-4-ylpyrazolo[1,5-a]pyrid-3-ylamine; pyrazolo[1,5-a]pyridine-3,5-diamine; 5-morpholin-4-ylpyrazolo[1,5-a]pyrid-3-ylamine; 2-[(3-aminopyrazolo[1,5-a]pyrid-5-yl)(2-hydroxyethyl)-amino]ethanol; 2-[(3-aminopyrazolo[1,5-a]pyrid-7-yl)(2-hydroxyethyl)amino]ethanol; 3-aminopyrazolo[1,5-a]pyrid-5-ol; 3-aminopyrazolo[1,5-a]pyrid-4-ol; 3-aminopyrazolo-[1,5-a]pyrid-6-ol; 3-aminopyrazolo[1,5-a]pyrid-7-ol; and addition salts thereof with an acid or with a base.

The pyrimidine derivatives may be chosen from the compounds described, for example, in patents DE 2,359,399; JP 88-169,571; JP 05-63,124; EP 0,770,375, and patent application WO 96/15765, such as 2,4,5,6-tetraminopyrimidine, 4-hydroxy-2,5,6-triaminopyrimidine, 2-hydroxy-4,5,6-triaminopyrimidine, 2,4-dihydroxy-5,6-diaminopyrimidine and 2,5,6-triaminopyrimidine, and pyrazolopyrimidine derivatives such as those disclosed in patent application FR-A-2,750,048, for example, pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)(2-hydroxyethyl)amino]ethanol, 2-[(7-aminopyrazolo[1,5-a]pyrimidin-3-yl)(2-hydroxyethyl)amino]ethanol, 5,6-dimethyl-pyrazolo[1,5-a]pyrimidine-3,7-diamine, 2,6-dimethylpyrazolo[1,5-a]pyrimidine-3,7-diamine, 2,5,N7,N7-tetramethylpyrazolo[1,5-a]pyrimidine-3,7-diamine and 3-amino-5-methyl-7-imidazolylpropylaminopyrazolo[1,5-a]pyrimidine, acid addition salts thereof and tautomeric forms thereof, when a tautomeric equilibrium exists.

The pyrazole derivatives that may be used herein include, for example, the compounds described in patents DE 3,843,892 and DE 4,133,957 and patent applications WO 94/08969, WO 94/08970, FR-A-2,733,749 and DE 19,543,988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl) pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and acid addition salts thereof.

When present, the additional oxidation bases are each generally present in an amount ranging from 0.001% to 10% by weight relative to the total weight of the dye composition, such as from 0.005% to 6% by weight relative to the total weight of the dye composition.

As indicated previously, the dye compositions may comprise at least one coupler.

These couplers include, for example, meta-phenylenediamines, meta-aminophenols, meta-diphenols, naphthalene-based couplers, heterocyclic couplers, for example, indole derivatives, indoline derivatives, sesamol and derivatives thereof, pyridine derivatives, pyrazolotriazole derivatives, pyrazolones, and the addition salts thereof.

Exemplary couplers include 2-methyl-5-aminophenol, 5-N-(β-hydroxyethyl)-amino-2-methylphenol, 6-chloro-2-methyl-5-aminophenol, 3-aminophenol, 1,3-dihydroxy-benzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis(β-hydroxyethylamino)toluene, and acid addition salts thereof.

In the compositions, the couplers are each generally present in an amount of ranging from 0.001% to 10% by weight relative to the weight of the dye composition, for example from 0.005% to 6% by weight relative to the weight of the dye composition.

The addition salts of the additional oxidation bases and couplers may be chosen from acid addition salts such as the hydrochlorides, hydrobromides, sulfates, citrates, succinates, tartrates, lactates, tosylates, benzenesulfonates, phosphates, and acetates.

The compositions optionally comprise at least one direct dye chosen from anionic, cationic, and neutral species such as a nonionic species.

Examples of direct dyes include nitrobenzene dyes, azo dyes, azomethine dyes, azine dyes, methine dyes, tetraazapentamethine dyes, quinone dyes, anthraquinone dyes, naphthoquinone dyes, benzoquinone dyes, phenothiazine dyes, indigoid dyes, indoamine dyes, xanthene dyes, phenanthridine dyes, phthalocyanin dyes, triarylmethane-based dyes, and natural dyes, alone or as mixtures.

Nitro chromophores that may be used include, for example, radicals derived from the following compounds:
1,4-diamino-2-nitrobenzene,
1-amino-2-nitro-4-(β-hydroxyethylamino)benzene,
1-amino-2-nitro-4-bis(β-hydroxyethyl)aminobenzene,
1,4-bis(β-hydroxyethylamino)-2-nitrobenzene,
1-β-hydroxyethylamino-2-nitro-4-bis-(β-hydroxyethylamino)benzene,
1-β-hydroxyethylamino-2-nitro-4-aminobenzene,
1-β-hydroxyethylamino-2-nitro-4-(ethyl)(β-hydroxyethyl)aminobenzene,
1-amino-3-methyl-4-β-hydroxyethylamino-6-nitrobenzene,
1-amino-2-nitro-4-β-hydroxyethylamino-5-chlorobenzene,
1,2-diamino-4-nitrobenzene,
1-amino-2-β-hydroxyethylamino-5-nitrobenzene,
1,2-bis(β-hydroxyethylamino)-4-nitrobenzene,
1-amino-2-[tris(hydroxymethyl)methylamino]-5-nitrobenzene,
1-hydroxy-2-amino-5-nitrobenzene,
1-hydroxy-2-amino-4-nitrobenzene,
1-hydroxy-3-nitro-4-aminobenzene,
1-hydroxy-2-amino-4,6-dinitrobenzene,
1-β-hydroxyethyloxy-2-β-hydroxyethylamino-5-nitrobenzene,
1-methoxy-2-β-hydroxyethylamino-5-nitrobenzene,
1-β-hydroxyethyloxy-3-methylamino-4-nitrobenzene,
1-β,γ-dihydroxypropyloxy-3-methylamino-4-nitrobenzene,
1-β-hydroxyethylamino-4-β,γ-dihydroxypropyloxy-2-nitrobenzene,
1-β,γ-dihydroxypropylamino-4-trifluoromethyl-2-nitrobenzene,
1-β-hydroxyethylamino-4-trifluoromethyl-2-nitrobenzene,
1-β-hydroxyethylamino-3-methyl-2-nitrobenzene,
1-β-aminoethylamino-5-methoxy-2-nitrobenzene,
1-hydroxy-2-chloro-6-ethylamino-4-nitrobenzene,
1-hydroxy-2-chloro-6-amino-4-nitrobenzene,
1-hydroxy-6-[bis(β-hydroxyethyl)amino]-3-nitrobenzene,
1-β-hydroxyethylamino-2-nitrobenzene, and
1-hydroxy-4-β-hydroxyethylamino-3-nitrobenzene.

Azo direct dyes, include, for example, azo dyes described in patent applications WO 95/15144, WO 95/01772, and EP 714 954 and the following dyes described in the Color Index International 3rd edition: Disperse Red 17, Acid Yellow 9, Acid Black 1, Basic Red 22, Basic Red 76, Basic Yellow 57, Basic Brown 16, Acid Yellow 36, Acid Orange 7, Acid Red 33, Acid Red 35, Basic Brown 17, Acid Yellow 23, Acid Orange 24, Disperse Black 9. Mention may also be made of 1-(4′-aminodiphenylazo)-2-methyl-4-[bis(β-hydroxyethyl)amino]benzene, and 4-hydroxy-3-(2-methoxyphenylazo)-1-naphthalenesulfonic acid.

Quinone dyes include, for example, radicals derived from the following dyes: Disperse Red 15, Solvent Violet 13, Acid Violet 43, Disperse Violet 1, Disperse Violet 4, Disperse Blue 1, Disperse Violet 8, Disperse Blue 3, Disperse Red 11, Acid Blue 62, Disperse Blue 7, Basic Blue 22, Disperse Violet 15, Basic Blue 99, and the following compounds: 1-N-methylmorpholiniumpropylamino-4-hydroxyanthraquinone, 1-amino-propylamino-4-methylaminoanthraquinone, 1-aminopropylaminoanthraquinone, 5-β-hydroxyethyl-1,4-diaminoanthraquinone, 2-aminoethylaminoanthraquinone, and 1,4-bis(β,γ-dihydroxypropylamino)anthraquinone.

Azine direct dyes include, for example, Basic Blue 17 and Basic Red 2.

Indoamine direct dyes include, for example, the following compounds: 2-β-hydroxyethylamino-5-[bis(β-4′-hydroxyethyl)amino]anilino-1,4-benzoquinone; 2-β-hydroxyethylamino-5-(2′-methoxy-4′-amino)anilino-1,4-benzoquinone; 3-N(2′-chloro-4′-hydroxy)phenylacetylamino-6-methoxy-1,4-benzoquinoneimine; 3-N(3′-chloro-4′-methylamino)phenylureido-6-methyl-1,4-benzoquinoneimine; and 3-[4′-N-(ethylcarbamylmethyl)amino]phenylureido-6-methyl-1,4-benzoquinoneimine.

The dyes described in U.S. Pat. No. 5,888,252, EP 1,133,975, WO 03/029359, EP 860,636, WO 95/01772, WO 95/15144, EP 714954, the encyclopedia “The Chemistry of Synthetic Dye” by K. Venkataraman, 1952, Academic Press, vol. 1 to 7, the encyclopedia “Kirk-Othmer” “Chemical Technology”, in the chapter “Dyes and Dye Intermediate”, 1993, Wiley & Sons, and in the encyclopedia “Ullmann's Encyclopaedia of Industrial Chemistry” 7th edition, Wiley & Sons may also be used.

When present, the amount of direct dyes in the composition generally ranges from 0.001% to 20% by weight relative to the total weight of the dye composition, and such as from 0.01% to 10% by weight relative to the total weight of the dye composition.

The medium suitable for dyeing, also known as the dye support, is a cosmetic medium that generally comprises water or comprises a mixture of water and at least one organic solvent to dissolve the compounds that would not be sufficiently soluble in water. Organic solvents, include, for example, of C₁-C₄ alkanols, such as ethanol and isopropanol; polyols and polyol ethers such as 2-butoxyethanol, propylene glycol, propylene glycol monomethyl ether, diethylene glycol monoethyl ether, and monomethyl ether; aromatic alcohols such as benzyl alcohol and phenoxyethanol; and mixtures thereof.

The solvents may be present in proportions ranging from approximately 1% to 40% by weight relative to the total weight of the dye composition, such as from 5% to 30% by weight.

The compositions may further comprise at least one oxidizing agent.

Oxidizing agents conventionally used for the oxidation dyeing of keratin fibers include, for example, hydrogen peroxide, urea peroxide, alkali metal bromates, persalts such as perborates and persulfates, peracids and oxidase enzymes, such as peroxidases, 2-electron oxidoreductases such as uricases, and 4-electron oxygenases, for instance laccases. For example, the oxidizing agent may be hydrogen peroxide.

The dye compositions may also further comprise various adjuvants conventionally used in hair dye compositions, such as anionic, cationic, nonionic, amphoteric, and zwitterionic surfactants and mixtures thereof; anionic, cationic, nonionic, amphoteric, and zwitterionic polymers and mixtures thereof; inorganic or organic thickeners, such as anionic, cationic, nonionic, and amphoteric associative polymeric thickeners; antioxidants; penetration agents; sequestering agents; fragrances; buffers; dispersing agents; packaging agents such as, optionally volatile and optionally modified silicones; film-forming agents; ceramides; preserving agents; and opacifiers.

The adjuvants are each generally present in an amount ranging from 0.01% to 20% by weight relative to the total weight of the composition.

A person skilled in the art can readily select this or these optional additional compounds such that the advantageous properties intrinsically associated with the oxidation dye compositions in accordance with the invention are not, or are not substantially, adversely affected by the additions envisaged.

The pH of the dye compositions generally ranges from 3 to 12, for example, from 5 to 11. It may be adjusted to the desired value using acidifying or basifying agents commonly used in the dyeing of keratin fibers or using standard buffer systems.

Acidifying agents include, for example, inorganic or organic acids such as hydrochloric acid; orthophosphoric acid; sulfuric acid; carboxylic acids such as acetic acid, tartaric acid, citric acid, and lactic acid; and sulfonic acids.

Basifying agents include, for example, aqueous ammonia, alkaline carbonates, alkanolamines such as mono-, di- and triethanolamine and derivatives thereof, sodium hydroxide, potassium hydroxide, and compounds of formula (II) below: 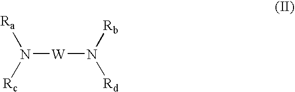
wherein W is a propylene residue that is unsubstituted or substituted with a hydroxyl group or a C₁-C₄ alkyl radical; Rₐ, R_{b}, R_{c} and Rd, which may be identical or different, are chosen from a hydrogen atom, a C₁-C₄ alkyl radical, and a C₁-C₄ hydroxyalkyl radical.

The dye compositions may be in various forms, such as in the form of liquids, creams, and gels, and in any other form that is suitable for dyeing keratin fibers, such as human hair.

The processes of the present invention are processes in which the compositions as described above are applied to wet or dry keratin fibers, such as human keratin fibers. The processes include processes for the oxidation dyeing of keratin fibers, wherein the dyeing compositions is applied to the wet or dry keratin fibers for a time sufficient to obtain a desired coloration of the fibers.

The oxidizing agents may be added to the dye compositions at the time of use, or it may be present in the oxidizing compositions, and applied simultaneously or sequentially to the dye compositions. In the latter case, the oxidizing agents are contained in compositions other than the dye compositions.

In some embodiments, the compositions are mixed, for example, at the time of use, with a composition comprising, in a medium that is suitable for dyeing, at least one oxidizing agent, present in an amount sufficient to obtain the desired coloration.

The mixture obtained is a ready-to-use composition and is then applied to the keratin fibers. After an action time ranging from 3 to 50 minutes, such as from 5 to 30 minutes, the keratin fibers are rinsed, optionally washed with shampoo and rinsed again, and then dried or allowed to dry.

The oxidizing composition may also comprise various adjuvants conventionally used in hair dyeing compositions and as defined above.

The pH of the oxidizing composition comprising the oxidizing agent is such that, after mixing with the dye composition, the pH of the resulting composition applied to the keratin fibers ranges from 3 to 12, such as from 5 to 11.

The pH may be adjusted to the desired value with acidifying or basifying agents usually used in the dyeing of keratin fibers and as described above.

The ready-to-use composition that is applied to the keratin fibers (the dye composition mixed with the composition comprising the oxidizing agent) may be in various forms, such as in the form of liquids, creams, or gels, or any other form that is suitable for dyeing keratin fibers, such as human hair.

In another aspect, multi-compartment dyeing devices or “kits”, are provided, in which a first compartment comprises a dye composition comprising the dye mixed as defined above and a second compartment comprises an oxidizing agent. The dye composition in the first compartment may be a lightening dye composition. The dye in the dye composition is optionally a cationic dye.

The devices may be equipped with a means for applying the desired mixture to the hair, such as the devices described in patent FR-2,586,913.

The invention is illustrated in greater detail by the non-limiting examples described below. Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific example are reported as precisely as possible. Any numerical value, however, inherently contains certain errors necessarily resulting from the standard deviation found in its respective testing measurements.

### EXAMPLES

### Example 1

### 1.A—Synthesis of N-methyl-N-(2-methylaminoethyl)benzene-1,4-diamine Dihydrochloride

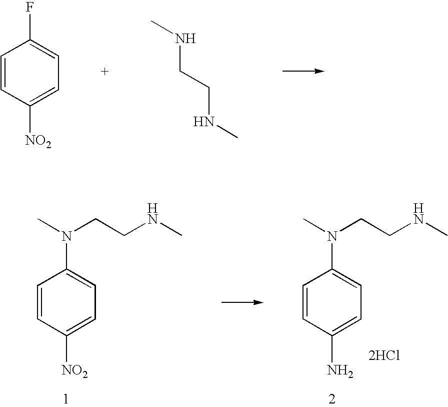

### Step 1: Synthesis of N,N″-dimethyl-N-(4-nitrophenyl)ethane-1,2-diamine 1

5 g of 4-fluoronitrobenzene (35 mmol) was dissolved in 50 ml of tetrahydrofuran (THF). 2 equivalents of N,N-dimethylenediamine was added dropwise. The reaction medium was maintained at the THF reflux temperature for 20 hours. The mixture was then poured onto crushed ice, and a precipitate formed. The precipitate was filtered off, washed with water and then dried.

### Step 2: Synthesis of N-methyl-N-(2-methylaminoethyl)benzene-1,4-diamine Dihydrochloride 2

The nitro compound 1 was dissolved in 500 ml of ethanol in a 1 L hydrogenator. 10% palladium on charcoal (50% water) was added and the hydrogenator was purged with nitrogen and then filled with hydrogen. After 1.5 hours, the palladium salts were filtered off. 20 ml of 3 M hydrochloric ethanol and then 300 ml of isopropyl ether were added. The precipitate obtained was filtered off and recrystallized from hydrochloric ethanol. After filtration and then drying, the product was obtained.

¹H NMR (ppm): 2.75 (s, 3H); 2.98 (s, 3H); 3.29 (t, 2H); 3.69 (t, 2H); 6.95 (m, 2H); 7.29 (m, 2H).

Mass: M=179

Elemental Analysis:

| | | | | |
|---|---|---|---|---|
| Theoretical: | C: 47.63 | H: 7.59 | N: 16.66 | Cl: 27.67 |
| Measured: | C: 47.04 | H: 7.69 | N: 15.75 | Cl: 27.67 |

### 1-B—Example of Dyeing

The dye composition below was prepared (contents in grams):

| Constituent | g % | |
|---|---|---|
| N-Methyl-N-(2-methylaminoethyl)benzene-1,4-diamine | 1.5 | |
| dichloride (6 × 10⁻³ mol) | | |
| 2,4-diaminophenoxyethanol dihydrochloride | 1.45 | |
| (Coupler (6 × 10⁻³ mol)) | | |
| Oleyl alcohol polyglycerolated with 2 mol of glycerol | 4 | |
| Oleyl alcohol polyglycerolated with 4 mol of glycerol | 5.69 | AM |
| Oleic acid | 3 | |
| Oleylamine containing 2 mol of ethylene oxide, sold under | 7 | |
| the trade name Ethomeen 012 by the company Akzo | | |
| Diethylaminopropyl laurylaminosuccinamate, sodium salt, | 3.0 | AM |
| containing 55% AM | | |
| Oleyl alcohol | 5 | |
| Oleic acid diethanolamide | 12 | |
| Ethyl alcohol | 7 | |
| Propylene glycol | 3.5 | |
| Dipropylene glycol | 0.5 | |
| Propylene glycol monomethyl ether | 9 | |
| Antioxidant/sequestering agent | qs | |
| Ammonium acetate | 0.8 | |
| Sodium metabisulfite as an aqueous 35% solution | 0.45 | |
| 20% aqueous ammonia | 10 | |
| Demineralized water, qs | 100 | g |

At the time of use, the above dye composition was mixed weight for weight with a 20-volumes hydrogen peroxide solution (6% by weight) of pH 3.

The mixture obtained was applied to locks of natural or permanent-waved grey hair, containing 90% white hairs, for 30 minutes. The locks were then rinsed, washed with a standard shampoo, rinsed again and then dried.

The shade obtained on the locks was a bright blue.

### Example 2

### 2.A—Synthesis of N-methyl-N-(6-methylaminohexyl)benzene-1,4-diamine (4)

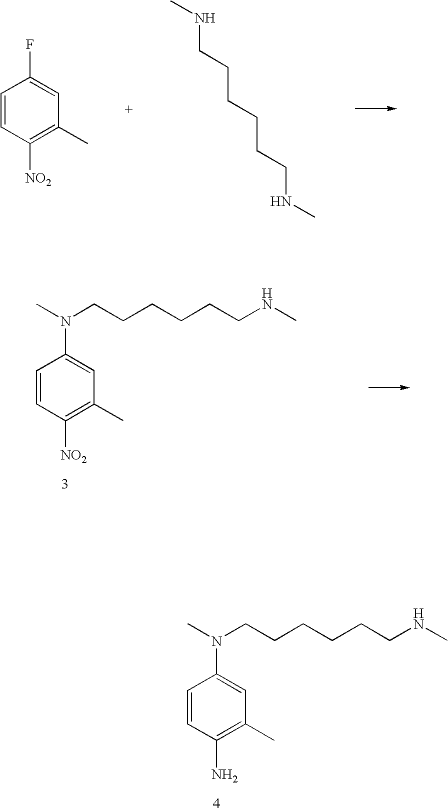

2 g of 4-fluoronitrobenzene (14 mmol) was dissolved in 8 ml of N-methylpyrrolidone. One equivalent of N,N′-dimethylhexane-1,6-diamine (1.86 g) and 2.90 g of potassium carbonate were added. The reaction medium was heated at 80° C. for 2 hours. The mixture was poured onto crushed ice, and a precipitate formed. The precipitate was filtered off, washed with water and then dried. The nitro compound 3 thus obtained was then reduced with zinc.

Mass: LC/MS ESI+: m/z=236 (M+1), 259 (M+1+23).

### 2.B—Examples of Dyeing in Acidic Medium

The following dye compositions were prepared:

| | Examples | | | | | | | |
|---|---|---|---|---|---|---|---|---|
| | 2.1 | | 2.2 | | 2.3 | | 2.4 | |
| N-Methyl-N-(6-methylaminohexyl) | 10⁻³ | mol | 10⁻³ | mol | 10⁻³ | mol | 10⁻³ | mol |
| benzene-1,4-diamine | | | | | | | | |
| 5-Amino-2-methylphenol | 10⁻³ | mol | | | | | | |
| 1H-Indol-6-ol | | | 10⁻³ | mol | | | | |
| 2-(2,4-Diaminophenoxy)ethanol | | | | | 10⁻³ | mol | | |
| hydrochloride | | | | | | | | |
| 3-Amino-2-chloro-6-methylphenol | | | | | | | 10⁻³ | mol |
| hydrochloride | | | | | | | | |
| Dye support (1) | (*) | | (*) | | (*) | | (*) | |
| Demineralized water qs | 100 | g | 100 | g | 100 | g | 100 | g |

(*) Dye support (1) pH 7

| | | |
|---|---|---|
| 96% ethyl alcohol | 20.8 | g |
| Sodium metabisulfite as an aqueous 35% solution | 0.23 | g AM |
| Pentasodium salt of diethylenetriaminepentaacetic acid, as | 0.48 | g AM |
| an aqueous 40% solution | | |
| C₈-C₁₀ alkyl polyglucoside as an aqueous 60% solution | 3.6 | g AM |
| Benzyl alcohol | 2.0 | g |
| Polyethylene glycol containing 8 ethylene oxide units | 3.0 | g |
| Na₂HPO₄ | 0.28 | g |
| KH₂PO₄ | 0.46 | g |

At the time of use, each composition was mixed with an equal weight of 20-volumes aqueous hydrogen peroxide solution (6% by weight). A final pH of 7 was obtained.

Each mixture obtained was applied to locks of grey hair containing 90% white hair. After leaving the mixture on for 30 minutes, the locks were rinsed, washed with a standard shampoo, rinsed again and then dried.

The dyeing results below were obtained.

Examples

| | 2.1 | 2.2 | 2.3 | 2.4 |
|---|---|---|---|---|
| Shade | grey | brown | strong blue- | blue |
| observed | | | green grey | |

### 2.C—Examples of Dyeing in Alkaline Medium

| | Examples | | | | | | | | | |
|---|---|---|---|---|---|---|---|---|---|---|
| | 2.5 | | 2.6 | | 2.7 | | 2.8 | | 2.9 | |
| N-Methyl-N-(6-methylamino- | 10⁻³ | mol | 10⁻³ | mol | 10⁻³ | mol | 10⁻³ | mol | 10⁻³ | mol |
| hexyl)benzene-1,4-diamine | | | | | | | | | | |
| 5-Amino-2-methylphenol | 10⁻³ | mol | | | | | | | | |
| 1H-Indol-6-ol | | | 10⁻³ | mol | | | | | | |
| 3,6-Dimethyl-1H-pyrazolo[5,1-c]- | | | | | 10⁻³ | mol | | | | |
| [1,2,4]triazole | | | | | | | | | | |
| 2-(2,4-Diaminophenoxy)ethanol | | | | | | | 10⁻³ | mol | | |
| hydrochloride | | | | | | | | | | |
| 3-Amino-2-chloro-6-methylphenol | | | | | | | | | 10⁻³ | mol |
| hydrochloride | | | | | | | | | | |
| Dye support (2) | (*) | | (*) | | (*) | | (*) | | (*) | |
| Demineralized water qs | 100 | g | 100 | g | 100 | g | 100 | g | 100 | g |

(*) Dye support (2) pH 9.5

| | | |
|---|---|---|
| 96° ethyl alcohol | 20.8 | g |
| Sodium metabisulfite as an aqueous 35% solution | 0.23 | g AM |
| Pentasodium salt of diethylenetriaminepentaacetic acid, as | 0.48 | g AM |
| an aqueous 40% solution | | |
| C₈-C₁₀ alkyl polyglucoside as an aqueous 60% solution | 3.6 | g AM |
| Benzyl alcohol | 2.0 | g |
| Polyethylene glycol containing 8 ethylene oxide units | 3.0 | g |
| NH₄Cl | 4.32 | g |
| Aqueous ammonia containing 20% NH₃ | 2.94 | g |

At the time of use, each composition was mixed with an equal weight of 20-volumes aqueous hydrogen peroxide solution (6% by weight). A final pH of 9.5 was obtained.

Each mixture obtained was applied to locks of grey hair containing 90% white hair. After leaving the mixture on for 30 minutes, the locks were rinsed, washed with a standard shampoo, rinsed again and then dried.

The dyeing results below were obtained.

| | Examples | | | | |
|---|---|---|---|---|---|
| | 2.5 | 2.6 | 2.7 | 2.8 | 2.9 |
| Shade | strong | orange | chromatic | blue- | blue |
| observed | blue-violet | | red-violet | green | |

### Example 3

### 3.A—Synthesis of N-(4-amino-3-methylphenyl)-N-methyl-N-[6-(methylamino)hexyl]amine (6)

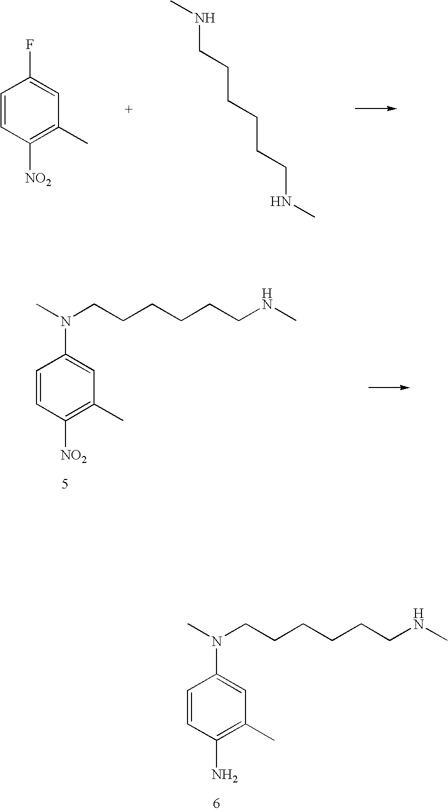

2 g of 4-fluoro-2-methyl-1-nitrobenzene (14 mmol) was dissolved in 8 ml of N-methylpyrrolidone. One equivalent of N,N′-dimethylhexane-1,6-diamine (1.86 g) and 2.90 g of potassium carbonate were added. The reaction medium was heated at 80° C. for 2 hours. The mixture was then poured onto crushed ice, and a precipitate formed. The precipitate was filtered off, washed with water and then dried. The nitro compound 5 thus obtained was then reduced with zinc.

Mass: LC/MS ESI+: m/z=250 (M+1), 273 (M+1+23).

### 3.B—Examples of Dyeing in Acidic Medium

The following dye compositions were prepared:

| | Examples | | | | | | | | | | | | | |
|---|---|---|---|---|---|---|---|---|---|---|---|---|---|---|
| | 3.1 | | 3.2 | | 3.3 | | 3.4 | | 3.5 | | 3.6 | | 3.7 | |
| N-(4-Amino-3-methylphenyl)- | 10⁻³ | mol | 10⁻³ | mol | 10⁻³ | mol | 10⁻³ | mol | 10⁻³ | mol | 10⁻³ | mol | 10⁻³ | mol |
| N-methyl-N-[6- | | | | | | | | | | | | | | |
| (methylamino)hexyl] | | | | | | | | | | | | | | |
| amine | | | | | | | | | | | | | | |
| Benzene-1,3-diol | 10⁻³ | mol | | | | | | | | | | | | |
| 5-Amino-2-methylphenol | | | 10⁻³ | mol | | | | | | | | | | |
| 1H-Indol-6-ol | | | | | 10⁻³ | mol | | | | | | | | |
| 2-Aminopyridin-3-ol | | | | | | | 10⁻³ | mol | | | | | | |
| 3,6-Dimethyl-1H-pyrazolo | | | | | | | | | 10⁻³ | mol | | | | |
| [5,1-c][1,2,4]triazole | | | | | | | | | | | | | | |
| 2-(2,4-Diaminophenoxy)- | | | | | | | | | | | 10⁻³ | mol | | |
| ethanol hydrochloride | | | | | | | | | | | | | | |
| 3-Amino-2-chloro-6-methylphenol | | | | | | | | | | | | | 10⁻³ | mol |
| hydrochloride | | | | | | | | | | | | | | |
| Dye support (1) | (*) | | (*) | | (*) | | (*) | | (*) | | (*) | | (*) | |
| Demineralized water qs | 100 | g | 100 | g | 100 | g | 100 | g | 100 | g | 100 | g | 100 | g |

At the time of use, each composition was mixed with an equal weight of 20-volumes aqueous hydrogen peroxide solution (6% by weight). A final pH of 7 was obtained.

Each mixture obtained was applied to locks of grey hair containing 90% white hair. After leaving the mixture on for 30 minutes, the locks were rinsed, washed with a standard shampoo, rinsed again and then dried.

The dyeing results below were obtained.

| | Examples | | | | | | |
|---|---|---|---|---|---|---|---|
| | 3.1 | 3.2 | 3.3 | 3.4 | 3.5 | 3.6 | 3.7 |
| Shade | yel- | strong | strong | brown | orange | strong | strong |
| ob- | low | violet- | grey | | | blue- | blue |
| served | | grey | | | | green | |

### 3.C—Examples of Dyeing in Alkaline Medium

| | Examples | | | | | | | | | | | | | |
|---|---|---|---|---|---|---|---|---|---|---|---|---|---|---|
| | 3.8 | | 3.9 | | 3.10 | | 3.11 | | 3.12 | | 3.13 | | 3.14 | |
| N-(4-Amino-3-methylphenyl)- | 10⁻³ | mol | 10⁻³ | mol | 10⁻³ | mol | 10⁻³ | mol | 10⁻³ | mol | 10⁻³ | mol | 10⁻³ | mol |
| N-methyl-N-[6- | | | | | | | | | | | | | | |
| (methylamino)hexyl]amine | | | | | | | | | | | | | | |
| Benzene-1,3-diol | 10⁻³ | mol | | | | | | | | | | | | |
| 5-Amino-2-methylphenol | | | 10⁻³ | mol | | | | | | | | | | |
| 1H-Indol-6-ol | | | | | 10⁻³ | mol | | | | | | | | |
| 2-Aminopyridin-3-ol | | | | | | | 10⁻³ | mol | | | | | | |
| 3,6-Dimethyl-1H-pyrazolo- | | | | | | | | | 10⁻³ | mol | | | | |
| [5,1-c][1,2,4]triazole | | | | | | | | | | | | | | |
| 2-(2,4-Diaminophenoxy)- | | | | | | | | | | | 10⁻³ | mol | | |
| ethanol hydrochloride | | | | | | | | | | | | | | |
| 3-Amino-2-chloro-6-methylphenol | | | | | | | | | | | | | 10⁻³ | mol |
| hydrochloride | | | | | | | | | | | | | | |
| Dye support (2) | (*) | | (*) | | (*) | | (*) | | (*) | | (*) | | (*) | |
| Demineralized water qs | 100 | g | 100 | g | 100 | g | 100 | g | 100 | g | 100 | g | 100 | g |

At the time of use, each composition was mixed with an equal weight of 20-volumes aqueous hydrogen peroxide solution (6% by weight). A final pH of 9.5 was obtained.

Each mixture obtained was applied to locks of grey hair containing 90% white hair. After leaving the mixture on for 30 minutes, the locks were rinsed, washed with a standard shampoo, rinsed again and then dried.

The dyeing results below were obtained.

| | Examples | | | | | | |
|---|---|---|---|---|---|---|---|
| | 3.8 | 3.9 | 3.10 | 3.11 | 3.12 | 3.13 | 3.14 |
| Shade | yellow- | strong | red | red- | strong | strong | strong |
| ob- | brown | violet | | brown | chro- | blue | blue- |
| served | | | | | matic | | violet |
| | | | | | red- | | |
| | | | | | violet | | |

1. A compound of formula (I): 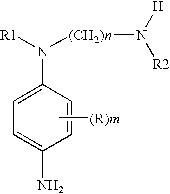
and addition salts thereof, wherein:
each R is independently chosen from linear and branched C₁-C₄ alkyl radicals; and linear and branched C₁-C₄ alkoxy radicals;
m is an integer ranging from 0 to 4;
R₁ is chosen from linear and branched C₁-C₄ alkyl radicals; linear and branched C₂-C₄ hydroxyalkyl radicals; and linear and branched (C₁-C₄)alkoxy(C₂-C₄)alkyl radicals;
R₂ is chosen from linear and branched C₁-C₄ alkyl radicals; linear and branched C₂-C₄ hydroxyalkyl radicals; and linear and branched (C₁-C₄)alkoxy(C₂-C₄)alkyl radicals; and
n is an integer ranging from 2 to 6.

2. A compound chosen from the following compounds and addition salts thereof:

| | |
|---|---|
| 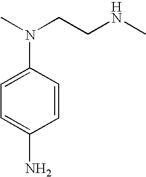 | N-Methyl-N-(2-methylaminoethyl)benzene-1,4-diamine |
| 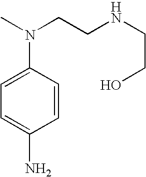 | 2-{2-[(4-Aminophenyl)methylamino]ethylamino}ethanol |
| 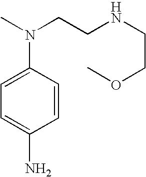 | N-[2-(2-Methoxyethylamino)ethyl]-N-methylbenzene-1,4- diamine |
| 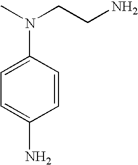 | N-(2-Aminoethyl)-N-ethylbenzene-1,4-diamine |
| 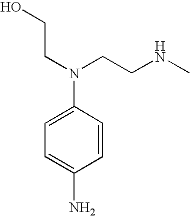 | 2-[(4-Aminophenyl)(2-methylaminoethyl)amino]ethanol |
| 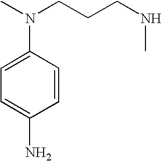 | N-Methyl-N-(3-methylaminopropyl)benzene-1,4-diamine |
| 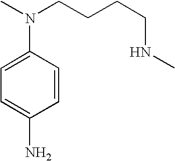 | N-Methyl-N-(4-methylaminobutyl)benzene-1,4-diamine |
| 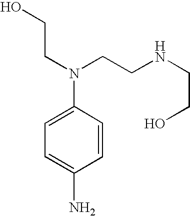 | 2-{(4-Aminophenyl)[2-(2-hydroxy- ethylamino)ethyl]amino}ethanol |
| 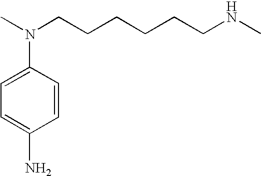 | N-Methyl-N-(6-methylaminohexyl)benzene-1,4-diamine |
| 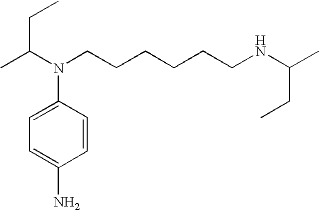 | N-sec-Butyl-N-(6-sec-butylaminohexyl)benzene-1,4- diamine |
| 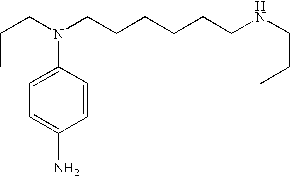 | N-Propyl-N-(6-propylaminohexyl)benzene-1,4-diamine |
| 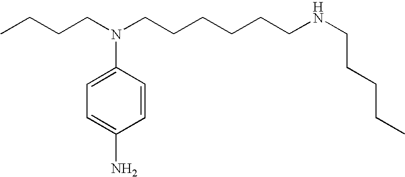 | N-Butyl-N-(6-butylaminohexyl)benzene-1,4-diamine |
| 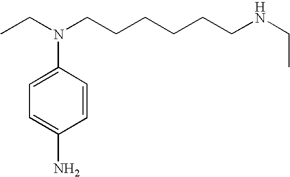 | N-Ethyl-N-(6-ethylaminohexyl)benzene-1,4-diamine |
| 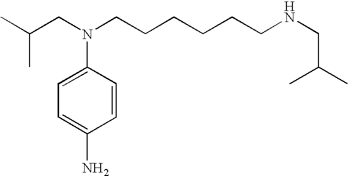 | N-Isobutyl-N-(6-isobutylaminohexyl)benzene-1,4-diamine |
| 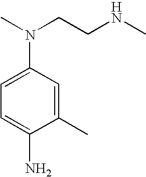 | 2,N′4′-Dimethyl-N4-(2-methylaminoethyl)benzene-1,4- diamine |
| 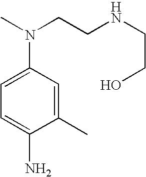 | 2-{2-[(4-Amino-3-methylphenyl)- methylamino]ethylamino}ethanol |
| 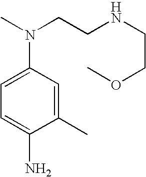 | N-4-[2-(2-Methoxyethylamino)ethyl]-2,N-4- dimethylbenzene-1,4-diamine |
| 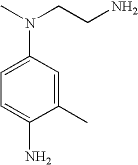 | N-4-(2-Aminoethyl)-N-4-ethyl-2-methylbenzene-1,4- diamine |
| 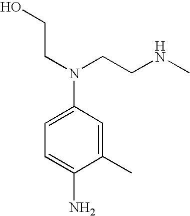 | 2-[(4-Amino-3-methylphenyl)(2- methylaminoethyl)amino]ethanol |
| 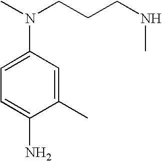 | 2,N-4-Dimethyl-N-4-(3-methylaminopropyl)benzene-1,4- diamine |
| 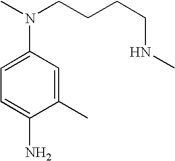 | 2,N-4-Dimethyl-N-4-(4-methylaminobutyl)benzene-1,4- diamine |
| 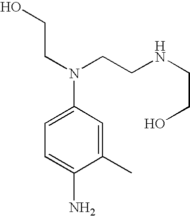 | 2-{(4-Amino-3-methylphenyl)[2-(2- hydroxyethylamino)ethyl]amino}ethanol |
| 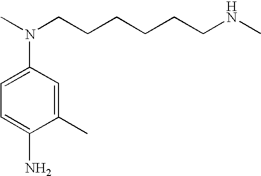 | 2,N-4-Dimethyl-N-4-(6-methylaminohexyl)benzene-1,4- diamine |
| 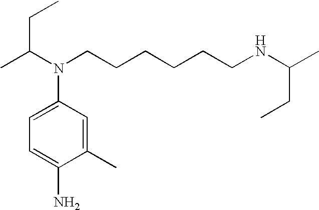 | N-4-sec-Butyl-N-4-(6-sec-butylaminohexyl)-2- methylbenzene-1,4-diamine |
| 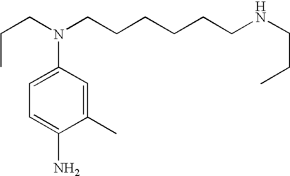 | 2-Methyl-N-4-propyl-N-4-(6-propylaminohexyl)benzene- 1,4-diamine |
| 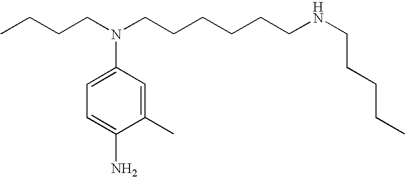 | N-4-Butyl-N-4-(6-butylaminohexyl)-2-methylbenzene-1,4- diamine |
| 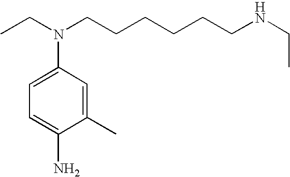 | N-4-Ethyl-N-4-(6-ethylaminohexyl)-2-methylbenzene-1,4- diamine |
| 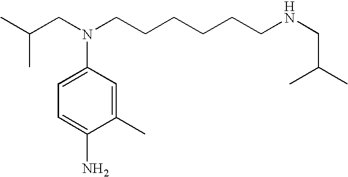 | N-4-Isobutyl-N-4-(6-isobutylaminohexyl)-2- methylbenzene-1,4-diamine |

3. A dye composition comprising, in a medium suitable for dyeing keratin fibers, at least one compound chosen from compounds of formula (I): 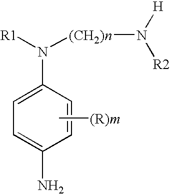
and addition salts thereof, wherein:
each R is independently chosen from linear and branched C₁-C₄ alkyl radicals; and
linear and branched C₁-C₄ alkoxy radicals;
m is an integer ranging from 0 to 4;
R₁ is chosen from linear and branched C₁-C₄ alkyl radicals; linear and branched C₂-C₄ hydroxyalkyl radicals; and linear and branched (C₁-C₄)alkoxy(C₂-C₄)alkyl radicals;
R₂ is chosen from a linear and branched C₁-C₄ alkyl radicals; linear and branched C₂-C₄ hydroxyalkyl radicals; and linear and branched (C₁-C₄)alkoxy(C₂-C₄)alkyl radicals; and
n is an integer ranging from 2 to 6.

4. The dye composition of claim 3, wherein the keratin fibers are human keratin fibers.

5. A dye composition comprising, in a medium suitable for dyeing keratin fibers, at least one compound chosen from the following compounds and addition salts thereof:

| | |
|---|---|
| 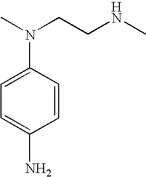 | N-Methyl-N-(2-methylaminoethyl)benzene-1,4-diamine |
| 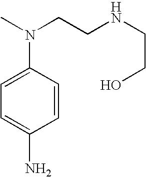 | 2-{2-[(4-Aminophenyl)methylamino]ethylamino}ethanol |
| 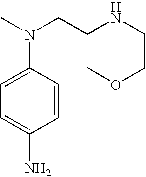 | N-[2-(2-Methoxyethylamino)ethyl]-N-methylbenzene-1,4- diamine |
| 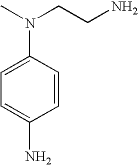 | N-(2-Aminoethyl)-N-ethylbenzene-1,4-diamine |
| 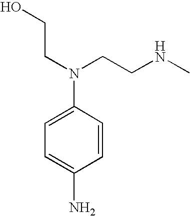 | 2-[(4-Aminophenyl)(2-methylaminoethyl)amino]ethanol |
| 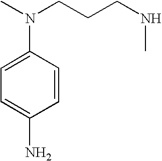 | N-Methyl-N-(3-methylaminopropyl)benzene-1,4-diamine |
| 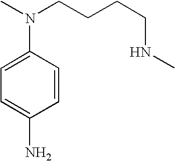 | N-Methyl-N-(4-methylaminobutyl)benzene-1,4-diamine |
| 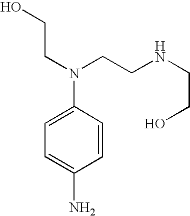 | 2-{(4-Aminophenyl)[2-(2-hydroxy- ethylamino)ethyl]amino}ethanol |
| 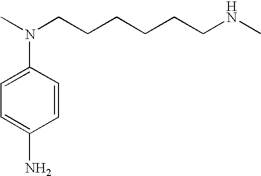 | N-Methyl-N-(6-methylaminohexyl)benzene-1,4-diamine |
| 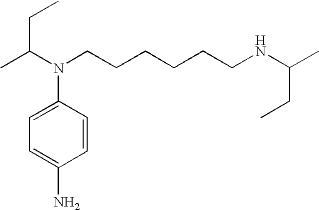 | N-sec-Butyl-N-(6-sec-butylaminohexyl)benzene-1,4- diamine |
| 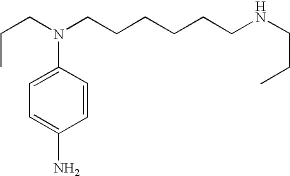 | N-Propyl-N-(6-propylaminohexyl)benzene-1,4-diamine |
| 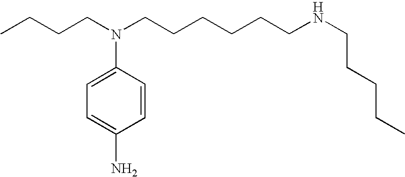 | N-Butyl-N-(6-butylaminohexyl)benzene-1,4-diamine |
| 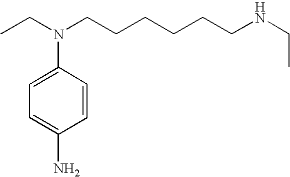 | N-Ethyl-N-(6-ethylaminohexyl)benzene-1,4-diamine |
| 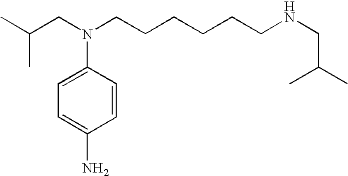 | N-Isobutyl-N-(6-isobutylaminohexyl)benzene-1,4-diamine |
| 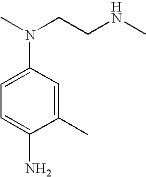 | 2,N′4′-Dimethyl-N4-(2-methylaminoethyl)benzene-1,4- diamine |
| 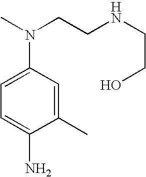 | 2-{2-[(4-Amino-3-methylphenyl)- methylamino]ethylamino}ethanol |
| 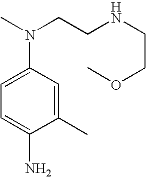 | N-4-[2-(2-Methoxyethylamino)ethyl]-2,N-4- dimethylbenzene-1,4-diamine |
| 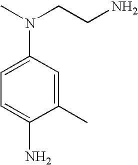 | N-4-(2-Aminoethyl)-N-4-ethyl-2-methylbenzene-1,4- diamine |
| 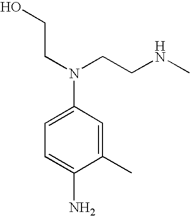 | 2-[(4-Amino-3-methylphenyl)(2- methylaminoethyl)amino]ethanol |
| 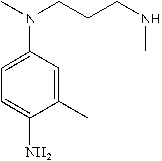 | 2,N-4-Dimethyl-N-4-(3-methylaminopropyl)benzene-1,4- diamine |
| 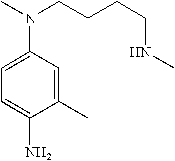 | 2,N-4-Dimethyl-N-4-(4-methylaminobutyl)benzene-1,4- diamine |
| 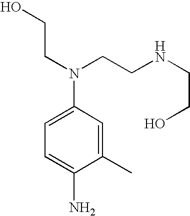 | 2-{(4-Amino-3-methylphenyl)[2-(2- hydroxyethylamino)ethyl]amino}ethanol |
| 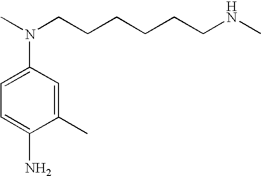 | 2,N-4-Dimethyl-N-4-(6-methylaminohexyl)benzene-1,4- diamine |
| 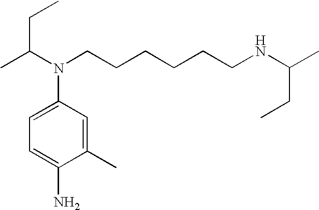 | N-4-sec-Butyl-N-4-(6-sec-butylaminohexyl)-2- methylbenzene-1,4-diamine |
| 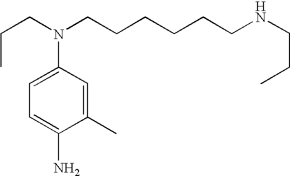 | 2-Methyl-N-4-propyl-N-4-(6-propylaminohexyl)benzene- 1,4-diamine |
| 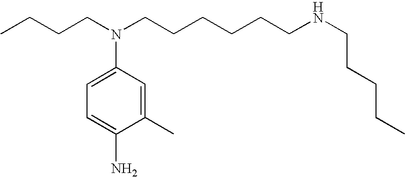 | N-4-Butyl-N-4-(6-butylaminohexyl)-2-methylbenzene-1,4- diamine |
| 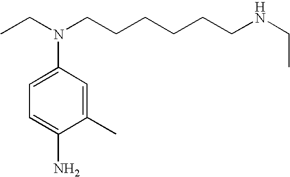 | N-4-Ethyl-N-4-(6-ethylaminohexyl)-2-methylbenzene-1,4- diamine |
| 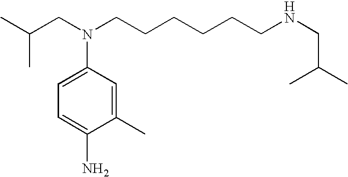 | N-4-Isobutyl-N-4-(6-isobutylaminohexyl)-2- methylbenzene-1,4-diamine |

6. The dye composition of claim 5, wherein the keratin fibers are human keratin fibers.

7. The dye composition according to claim 3, wherein the compound of formula (I) is present in an amount ranging from 0.001% to 10% by weight relative to the total weight of the dye composition.

8. The dye composition according to claim 7, wherein the compound of formula (I) is present in an amount ranging from 0.005% to 6% by weight relative to the total weight of the dye composition.

9. The dye composition according to claim 3, further comprising at least one additional oxidation base optionally combined with at least one coupler.

10. The dye composition according to claim 9, wherein the additional oxidation base is chosen from para-phenylenediamines other than the compounds of formula (I), bis(phenyl)alkylenediamines, para-aminophenols, bis-para-aminophenols, ortho-aminophenols and heterocyclic bases, and addition salts thereof.

11. The dye composition according to claim 9, wherein the additional oxidation base is present in an amount ranging from 0.001% to 10% by weight relative to the total weight of the dye composition.

12. The composition according to claim 9, wherein the at least one coupler is chosen from meta-phenylenediamines, meta-aminophenols, meta-diphenols, naphthalene-based couplers, and heterocyclic couplers, and addition salts thereof.

13. The composition according claim 9, wherein the at least one coupler is present in an amount ranging from 0.001% to 10% by weight relative to the total weight of the dye composition.

14. The composition according to claim 3, further comprising at least one direct dye chosen from neutral, anionic and cationic direct dyes.

15. The composition according to claim 14, wherein the at least one direct dye is chosen from nitrobenzene dyes, azo dyes, azomethine dyes, azine dyes, methine dyes, tetraazapentamethine dyes, quinone dyes, anthraquinone dyes, naphthoquinone dyes, benzoquinone dyes, phenothiazine dyes, indigoid dyes, indoamine dyes, xanthene dyes, phenanthridine dyes, phthalocyanin dyes, triarylmethane-based dyes, natural dyes, and mixtures thereof.

16. The composition according to claim 14, wherein the at least one direct dye is present in an amount ranging from 0.001% to 20% by weight relative to the total weight of the dye composition.

17. The composition according to claim 16, wherein the at least one direct dye is present in an amount ranging from 0.01% to 10% by weight relative to the total weight of the dye composition.

18. The composition according to claim 3, further comprising at least one oxidizing agent.

19. The composition according to claim 18, wherein the at least one oxidizing agent is chosen from hydrogen peroxide, urea peroxide, alkali metal bromates, persalts, peracids, and enzymes.

20. The composition according to claim 19, wherein the persalts are chosen from perborates and persulfates and the enzymes are chosen from peroxidases and 2- or 4-electron oxidoreductases.

21. The composition according to claim 19, wherein the at least one oxidizing agent is hydrogen peroxide.

22. A process for oxidation dyeing of keratin fibers, comprising applying a dye composition to the keratin fibers, which may be wet or dry, for a time sufficient to obtain a desired coloration,
wherein the dye composition comprises, in a medium suitable for dyeing keratin fibers, at least one compound chosen from compounds of formula (I): 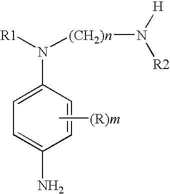
and addition salts thereof, wherein:
each R is independently chosen from linear and branched C₁-C₄ alkyl radicals; and linear and branched C₁-C₄ alkoxy radicals;
m is an integer ranging from 0 to 4;
R₁ is chosen from linear and branched C₁-C₄ alkyl radicals; linear and branched C₂-C₄ hydroxyalkyl radicals; and linear and branched (C₁-C₄)alkoxy(C₂-C₄)alkyl radicals;
R₂ is chosen from linear and branched C₁-C₄ alkyl radicals; linear and branched C₂-C₄ hydroxyalkyl radicals; and linear and branched (C₁-C₄)alkoxy(C₂-C₄)alkyl radicals; and
n is an integer ranging from 2 to 6.

23. The process of claim 22, wherein the keratin fibers are human keratin fibers.

24. The process according to claim 22, wherein at least one oxidizing agent is applied to the fibers in the form of at least one oxidizing composition simultaneously with or sequentially to the dye compositions.

25. A multi-compartment device comprising a first compartment comprising a dye composition and a second compartment comprising an oxidizing agent,
wherein the dye composition comprises, in a medium suitable for dyeing keratin fibers, at least one compound chosen from compounds of formula (I): 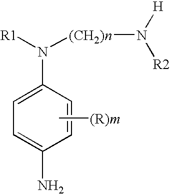
and addition salts thereof, wherein:
each R is independently chosen from linear and branched C₁-C₄ alkyl radicals; and linear and branched C₁-C₄ alkoxy radicals;
m is an integer ranging from 0 to 4;
a R₁ is chosen from linear and branched C₁-C₄ alkyl radicals; linear and branched C₂-C₄ hydroxyalkyl radicals; and linear and branched (C₁-C₄)alkoxy(C₂-C₄)alkyl radicals;
R₂ is chosen from linear and branched C₁-C₄ alkyl radicals; linear and branched C₂-C₄ hydroxyalkyl radicals; and linear and branched (C₁-C₄)alkoxy(C₂-C₄)alkyl radicals; and
n is an integer ranging from 2 to 6.
